(19) United States
(12) Patent Application Publication
(10) Pub. No.: US 2014/0135917 A1
(43) Pub. Date: May 15, 2014

(54) MULTI-FOCUS INTRAOCULAR PROSTHESIS

(71) Applicant: ALAN N. GLAZIER, Rockville, MD (US)

(72) Inventor: ALAN N. GLAZIER, Rockville, MD (US)

(73) Assignee: VISION SOLUTIONS TECHNOLOGIES, INC., ROCKVILLE, MD (US)

(21) Appl. No.: 13/804,204

(22) Filed: Mar. 14, 2013

Related U.S. Application Data
(60) Provisional application No. 61/725,855, filed on Nov. 13, 2012.

(51) Int. Cl. A61F 2/16

(52) U.S. Cl. CPC A61F 2/1635
USPC 623/6.13

(57) ABSTRACT

A multi-focus intraocular prosthesis is provided that makes use of fluid substitution to change the power of the prosthesis. Also provided are methods of making and using the same.

### CROSS-REFERENCE TO RELATED APPLICATION(S)

This invention claims the benefit of priority of U.S. Provisional Application No. 61/725,855 of Alan N. Glazier entitled “Multi-Focus Intraocular Lens” filed Nov. 13, 2012, the complete disclosure of which is incorporated herein by reference.

### FIELD OF THE INVENTION

The invention relates generally to a prosthesis and the use and production of a prosthesis for treatment of and surgical procedures involving the eyes, including but not limited to aphakia, pseudophakia, anterior cortical cataract extraction (acce), posterior cortical cataract extraction (pcce), accommodative restorative surgery for presbyopes, refractive correction surgery, and retinal degenerative conditions (e.g., low vision, macular degeneration).

### BACKGROUND

Light entering the emmetropic human eye is converged towards a point focus on the retina known as the fovea. The cornea and tear film are responsible for the initial convergence of entering light. Subsequent to corneal refraction, the incoming light passes through the physiological crystalline lens, where the light is refracted towards a point image on the fovea. The amount of bending to which the light is subjected is termed the refractive power. The refractive power needed to focus on an object depends upon how far away the object is from the principal planes of the eye. More refractive power is required for converging light rays to view close objects with clarity than is required for viewing distant objects with clarity.

A young and healthy physiological lens of the human eye has sufficient elasticity to permit its deformation by a process known as accommodation. The term accommodation refers to the ability of the eye to adjust focus between the distant point of focus, called the punctum remotum or pr (far point beyond 20 feet or 6 meters away), and the near point of focus called the punctum proximum or pp (near point within 20 feet or 6 meters away from the eye). The convexity of the lens decreases for far vision and increases for near vision so that the incoming light rays from the pr and pp are focused on or “coincident” with the retina.

Presbyopia is an age-related condition whereby incoming light rays from the pp are focuses at a virtual point situated behind the retina. According to one theory behind presbyopia, the physiological crystalline lens slowly loses its elasticity as it ages. Eventually, the crystalline lens lacks sufficient flexibility to obtain the convexity needed for near-point focus. According to another theory, the physiological lens enlarges with age and causes a decrease in working distance between the lens and the retina, resulting in decreased focus ability for the same muscle action. For most people, it becomes necessary around the age of 40-45 to use near addition lenses such as eyeglasses to artificially regain sufficient amplitude at near to accommodate for the pp when attempting to perform near-point activities such as reading. Once corrected, distance and near objects can be seen clearly.

Another condition of aging that can adversely affect vision is the formation of a cataract, which is the clouding of the crystalline lens. Cataracts can occur in either or both eyes. Cataracts are typically treated using a surgical procedure whereby the crystalline lens is replaced with a synthetic intraocular lens. However, current synthetic intraocular lenses lack the flexibility of a physiological crystalline lens to allow for near-vision accommodation. As a consequence, it is difficult, if not impossible, to focus a synthetic intraocular lens in the same way as a physiological lens to adjust for objects near the pp. Thus, conventional intraocular lenses are mostly monofocal and provide little, if any accommodating ability. As with presbyopia, patients of cataract surgery may use a plus-powered eyeglass lens to adjust vision for objects near the pp. Generally, a lens in front of their eye requires the equivalent of approximately +2.50 diopters of power to be able to focus on near-point objects between approximately 12 and 20 inches from the eye. However, “reading” glasses and contact lenses have the drawbacks of being inconvenient, uncomfortable, susceptible to loss and breakage, and in the case of glasses, aesthetically undesirable to many users.

Another problem that may adversely affect an individual's eyesight, both near and far, is retinal degenerative condition (RDC). Generally, a RDC involves damage to the macula. A RDC such as macular degeneration leaves the afflicted individual with a “blind spot” or scotoma usually at or near the center of a person's visual field. The afflicted individual is often only able to see peripheral images outside the blind spot. The visual field provided by such peripheral images is often insufficient to allow the individual to perform routine activities such as reading, driving a vehicle, or even daily chores and errands.

A person who suffers from a RDC is typically treated optically by using magnification or prism in lens form. A Galilean telescopic magnifying device may be placed in front of the eye or in the eye and customized to the user's needs. The magnification of the device enlarges the image viewed, expanding the image into healthier areas of retina peripheral (eccentric) to the scotoma. At near, the person suffering from a RDC usually needs magnification in the form of magnifying plus powered lenses and/or prisms—the former (i.e., the plus lenses and magnifiers) to help enlarge the image outside of the scotoma as in the telescopic example and the latter (e.g., the prisms) to help shift the images to different, more functional areas of the retina.

### SUMMARY

According to a first aspect of the invention, a multi-focus intraocular prosthesis is provided that includes a lens body having a chamber and first and second fluids in the chamber. Tilting movement of the lens body induces fluid substitution between the first and second fluids in an optical zone portion of the lens body.

A second aspect of the invention provides a multi-focus intraocular prosthesis including a lens body configured for placement in an eye to replace or supplement a physiological or artificial lens, and a plurality of fluids. The lens body has an optical axis and includes a transparent anterior wall member and a transparent posterior wall member, the anterior wall member and the posterior wall member having respective inner surfaces that collectively establish a chamber within the lens body. The chamber has an optical zone portion intersected by the optical axis, a substantially annular non-optical zone portion peripherally arranged radially outside the optical zone portion and in fluid communication with the optical zone portion, and a detainment structure. The plurality of fluids include a first fluid having a first refractive index and a first specific density and a second fluid having a second refractive index and a second specific density that differ from the first refractive index and the first specific density, respectively, the first and second fluids being immiscible with one another. The first fluid substantially fills the optical zone portion and the second fluid is situated substantially outside of the optical zone portion in the non-optical zone portion at a straight ahead gaze position in which the optical axis is horizontal. The second fluid substantially fills the optical zone portion and the first fluid is situated substantially outside of the optical zone portion in the non-optical zone portion at a downward gaze position in which the optical axis is at a tilt angle relative to horizontal of greater than zero but less than 90 degrees.

A third aspect of the invention provides a multi-focus intraocular prosthesis featuring a lens body and a plurality of fluids. The lens body is configured for placement in an eye to replace or supplement a physiological or artificial lens. The lens body has an optical axis and includes a transparent anterior wall member and a transparent posterior wall member, the anterior wall member and the posterior wall member having respective inner surfaces that collectively establish a chamber within the lens body. The chamber includes an optical zone portion intersected by the optical axis, and a substantially annular non-optical zone portion peripherally arranged relative to the optical zone portion and in fluid communication with the optical zone portion. The plurality of fluids includes first and second fluids in the chamber. The first fluid has a first refractive index and a first specific density and a second fluid has a second refractive index and a second specific density that differ from the first refractive index and the first specific density, respectively, the first and second fluids being immiscible with one another. The first fluid substantially fills the optical zone portion and the second fluid is situated substantially outside of the optical zone portion in the non-optical zone portion at a straight ahead gaze position in which the optical axis is horizontal. The second fluid substantially fills the optical zone portion and the first fluid is situated substantially outside of the optical zone portion in the non-optical zone portion at a downward gaze position in which the optical axis is at a tilt angle relative to horizontal of greater than zero but less than 90 degrees. Fluid substitution of the second fluid for the first fluid in the optical zone portion occurs at the tilt angle.

According to a fourth aspect of the invention, a method of making a multi-focus intraocular prosthesis, such as the multi-focus intraocular prostheses of the first, second and third aspects, is provided.

A fifth aspect of the invention provides a method of using a multi-focus intraocular prosthesis, for example, for treatment of and surgical procedures involving the eyes, including but not limited to aphakia, pseudophakia, anterior cortical cataract extraction (acce), posterior cortical cataract extraction (pcce), accommodative restorative surgery for presbyopes, refractive correction surgery, and retinal degenerative conditions (e.g., low vision, macular degeneration).

It is to be understood that the aspects described above are not exclusive or exhaustive of the scope of the invention. This invention encompasses other prostheses, intraocular lenses, devices, systems, kits, combinations, and methods/processes of making and using the same.

### BRIEF DESCRIPTION OF THE DRAWINGS

The accompanying drawings are incorporated in and constitute a part of the specification. The drawings, together with the general description given above and the detailed description of the exemplary embodiments and methods given below, serve to explain the principles of the invention. In such drawings:

FIG. 1 is a plan view of a multi-focus intraocular prosthesis according to an embodiment of the invention;

FIG. 2 is a side view of the multi-focus intraocular prosthesis of FIG. 1;

FIG. 3 is a side sectional view of the lens body (without haptics for simplicity) of the multi-focus intraocular prosthesis of FIGS. 1 and 2 taken along sectional line III-III of FIG. 1;

FIGS. 4A, 5 and 6A are cross-sectional views collectively illustrating fluid movement in the multi-focus intraocular prosthesis of FIGS. 1-3 during a downward progression of movements starting at a straight ahead gaze position (FIG. 4A) to an angled downward gaze position (FIG. 5) to a vertically downward gaze position (FIG. 6);

FIGS. 4B and 6B are cross-sectional views taken along sectional lines IVB-IVB and VIB-VIB of FIGS. 4A and 6A, respectively;

FIGS. 7-10 are cross-sectional views collectively illustrating fluid movement in the multi-focus intraocular prosthesis of FIGS. 1-3 during an upward progression of movements starting at a 90-degree vertically downward gaze position (FIG. 7) to a first angled downward gaze position (FIG. 8) to a second angled downward gaze position (FIG. 9) to a straight ahead gaze position (FIG. 10);

FIG. 11 is a cut-away isometric view of a multi-focus intraocular prosthesis according to another embodiment of the invention;

FIG. 12 is a plan view of a multi-focus intraocular prosthesis according to yet another embodiment of the invention;

FIG. 13 is a side view of the multi-focus intraocular prosthesis of FIG. 12;

FIG. 14 is a cross-sectional view taken along sectional line XIV-XIV of FIG. 12, showing the multi-focus intraocular prosthesis of FIG. 12 in straight-ahead gaze position;

FIG. 15 is a cross-sectional view taken along sectional line XV-XV of FIG. 12, showing the multi-focus intraocular prosthesis of FIG. 12 in vertically downward gaze position;

FIG. 16 is a perspective view of the multi-focus intraocular prosthesis of FIG. 12;

FIGS. 17A, 18 and 19A are cross-sectional views collectively illustrating fluid movement in a multi-focus intraocular prosthesis according to still another embodiment of the invention during a downward progression of movements starting at a straight ahead gaze position (FIG. 17A) to an angled downward gaze position (FIG. 18) to a vertically downward gaze position (FIG. 19A); and

FIGS. 17B and 19B are cross-sectional views taken along sectional lines XVIIB-XVIIB and XIXB-XIXB of FIGS. 17A and 19A, respectively.

### DESCRIPTION OF EXEMPLARY EMBODIMENTS

Reference will now be made in detail to the presently exemplary embodiments and methods of the invention as illustrated in the accompanying drawings, in which like reference characters designate like or corresponding parts throughout the drawings. It should be noted, however, that the invention in its broader aspects is not limited to the specific details, representative devices and methods, and illustrative examples shown and described in this section in connection with the exemplary embodiments and methods. The invention according to its various aspects is particularly pointed out and distinctly claimed in the attached claims read in view of this specification, and appropriate equivalents.

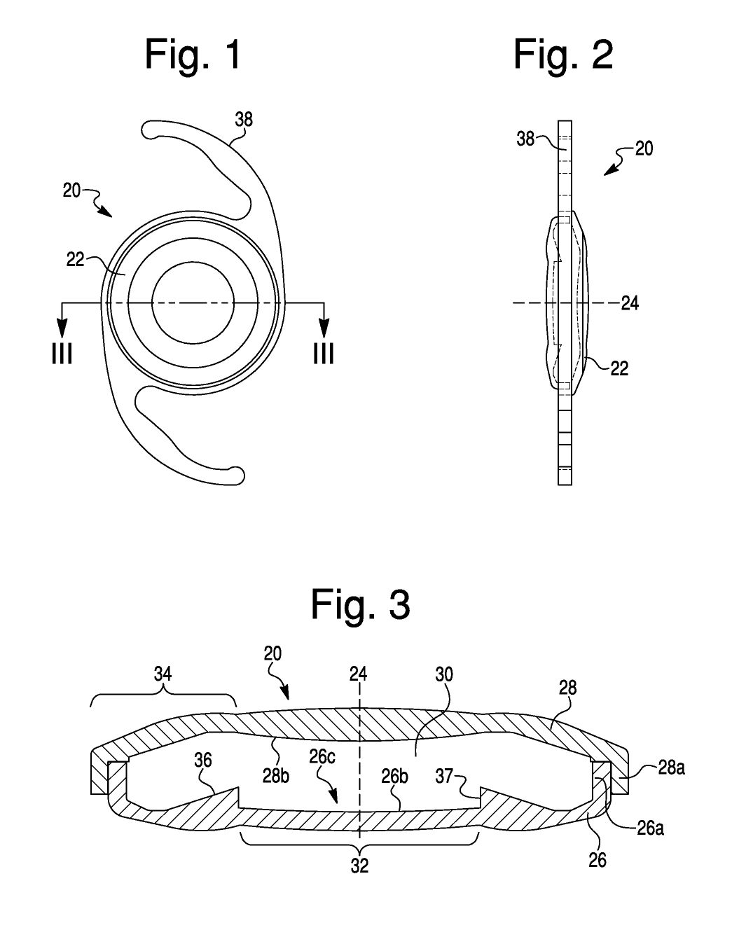

A multi-focus intraocular lens according to an embodiment of the invention is generally designated by reference numeral 20 in FIGS. 1-3. The intraocular lens 20 includes a lens body 22 sized and configured for placement in an eye of a human or animal to replace or supplement a physiological or artificial lens. The intraocular lens 20 also includes haptics 38 extending outward from diametrically opposite sides of the lens body 22. The haptics 38 may be integrally formed with the lens body 22 to establish a unitary, monolithic body, as described further below. Alternatively, the haptics 38 may be fastened, welded, fused, adhered and/or otherwise joined to the lens body 22. As generally understood in the art, haptics 38 may serve to secure or anchor the lens body 22 to a physiological structure of the eye. It should be understood that intraocular lens 20 may include alternative securing parts or mechanisms, such as the “Iris claw.”

The lens body 22 has an optical axis 24 concentric to the lens body 22. As best shown in FIG. 3, the lens body 22 includes a transparent anterior wall member 26 and a transparent posterior wall member 28. The anterior wall member 26 and the posterior wall member 28 are substantially disc-shaped with peripheral flange portions 26a and 28a, respectively. The flange portions 26a, 28a each extend substantially parallel to the optical axis 24. The peripheral flange 28a of the posterior wall member 28 is concentric with and circumferentially surrounds the peripheral flange 26a of the anterior wall member 26 so that the radially outer surface of the flange 26a abuts the radially inner surface of the flange 28a. The flanges 26a and 28a may be pressure fitted together and sealed to one another, for example, by fastening, welding, fusing, adhering and/or otherwise joining Alternatively, the anterior and posterior wall members 26 and 28 may be molded as a unitary or integral member. Although not shown, the embodiment of FIGS. 1-3 may be modified so that the peripheral flange 26a is positioned outward of and surrounds the peripheral flange 28a.

The central regions of the anterior wall member 26 and the posterior wall member 28 corresponding to an optical zone portion (or zone) 32 constitute or include optic elements or optics. Generally, when the intraocular lens 20 is implanted into its subject, such as a human or animal, especially mammals, it is the optical zone portion 32 through which the incoming light rays pass and converge on the retina. The chamber 30 also includes a non-optical zone portion 34 positioned radially outward of the periphery of the optical zone portion 32. In FIGS. 1-3, the non-optical zone portion 34 is substantially annular and surrounds the optical zone portion 32.

In the illustrated embodiment of FIGS. 1-3, the anterior wall member 26 incorporates an integral optic element having a convex-concave shape, and the posterior wall member 28 incorporates an integral optic element having a convex-convex shape. Alternative combinations may be selected depending upon the desired effective power and refractive properties of the intraocular lens 20, e.g., concave-convex, concave-concave, etc. Additionally, the interior and/or exterior surface(s) of the optics of the anterior wall member 26 and the posterior wall member 28 may have a non-curved or flat surface with a radius of curvature equal to zero, e.g., convex-flat, flat-convex, concave-flat, or flat-concave. The anterior and posterior wall members 26 and 28 may provide any combination of positive, negative, or no power optics. It is also possible to use laminates as optic elements, and to employ lenses with discrete refractive zones, especially concentric zones, such as in the case of Fresnel magnification. These are just some of the variations and modifications envisioned and encompassed herein. While much of the specification is described in reference to a human subject, it should be understood that the subject may be an animal, particularly a mammal, for example, for testing or veterinarian purposes.

The anterior wall member 26 and the posterior wall member 28 have respective interior surfaces 26b and 28b that collectively establish a chamber 30 within the lens body 22. The anterior wall member 26 includes a flow-delaying, detainment structure 36. In FIG. 3, the detainment structure 36 is depicted as an annular ridge immediately outside (radially) of the optical zone portion 32. The detainment structure 36, which is embodied as a ridge in the case of FIGS. 1-3, terminates radially inward at an annular shoulder 37 that defines a peripheral wall of an open pocket or cavity 26c at the center of the interior surface 26b. The optical axis 24 is substantially concentric with the central cavity 26c.

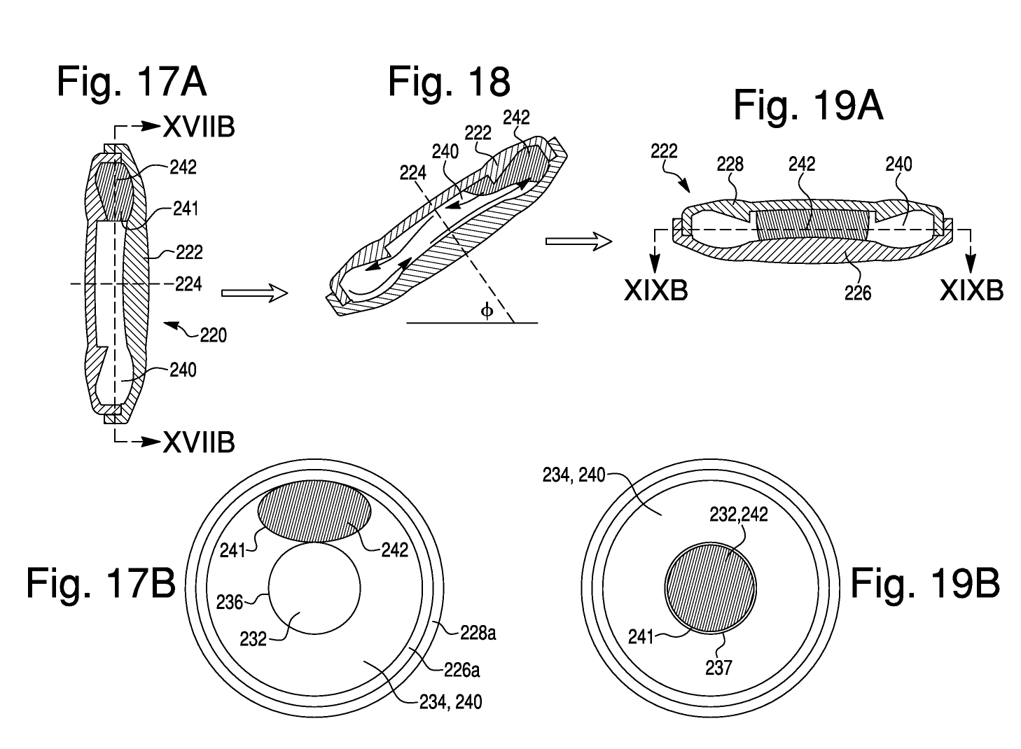

At its peak, the detainment structure/ridge 36 and the opposite portion of the interior surface 28b establish an annular constricted passage fluidly connecting the optical zone portion 32 of the chamber 30 with the non-optical zone portion 34 of the chamber 32. The ridge 36 tapers in height from the top of the annular shoulder 37 in a radially outward direction, as best shown in FIG. 3. It should be understood that the detainment structure 36 may be embodied as structures other than a tapering ridge. For example, the detainment structure 36 may be non-tapering, such as a wall, barrier, or other protrusion. Although the detainment structure 36 of FIGS. 1-3 constitutes part of the interior surface 26b of the anterior wall member 26, instead the detainment structure 36 may constitute part of the interior surface 28b of the posterior wall member 28, as discussed in further detail below with respect to the embodiment illustrated in FIGS. 17A, 17B, 18, 19A, and 19B. Alternatively, the interior surfaces 26b, 28b of both the anterior and posterior wall members 26, 28 may possess ridges or other detainment structures that cooperate with one another to form the constricted passage. In still another embodiment, the ridge may be excluded, such that the detainment structure 36 is the cavity or pocket 26c recessed into the anterior wall member 26 without a surrounding annular ridge.

In the illustrated embodiment, the cavity/pocket 26c is substantially commensurate in diameter with the perimeter of the optical zone portion 32 of the chamber 30. The optical zone portion 32 is intersected by the optical axis 26 and is generally centered in the lens body 22. The detainment structure 36 forms the constricted passage at the interface of the optical zone portion 32 and the non-optical zone portion 34 of the chamber 30. The constricted passage at the apex of the ridge 36 is sufficient in thickness (or height, as viewed in FIG. 3) to permit fluid communication the optical zone portion 32 and the non-optical zone portion 34 of the chamber 30, such that fluid substitution may take place between the optical zone portion 32 and the non-optical zone portion 34, and vice versa, depending upon the orientation of the prosthesis 20, as discussed in greater detail below.

The detainment structure 36 affects the flow of fluids in the chamber 30, and more particularly the substation of fluids into and out of the optical zone portion 32. As described below, the detainment structure 36, including the shoulder 37, temporarily captures one of the fluids in the optical zone portion 32 to delay the start of the fluid substitution, i.e., the exchange of the fluid in the optical zone portion 32 with the fluid in the non-optical zone portion 34. The thickness of the chamber 30 (that is, the distance by which the opposite interior surfaces 26b and 28b are spaced apart from one another) is greater at the central cavity 26c than at the constricted passage. The constricted passage established by the detainment structure 36 is illustrated as an annular gap extending 360 degrees about the periphery of the optical zone portion 32. Although not shown, the constricted passage may be non-continuous. For example, the detainment structure 36 may include “bridges” spanning between the interior surfaces 26b, 28b so that the constricted passage comprises multiple non-continuous fenestrations spaced from one another.

In the intraocular lens 20 illustrated in FIGS. 1-3, the chamber 30 defined between the anterior and posterior wall members 26 and 28 of the intraocular lens 20 is free of (that is, without) interior or exterior elongate channels and tubes, particularly between the optical zone portion 32 and the surrounding non-optical zone portion 34, that might prevent deforming or folding of the lens body 22 during surgical implantation. Further, no internal plate, lens, or other structure is situated between the anterior and poster wall members 26 and 28 in the illustrated embodiment of FIGS. 1-3. However, it is possible (although not shown) to provide one or both of the haptics 38 with a channel or channels that are in fluid communication with the chamber 30.

The intraocular lens 20 may be implanted in the posterior chamber of the eye so as to replace or supplement the crystalline lens. The intraocular lens 20 is arranged in the eye so that the optical axis 24 extends along the path of light that is refracted by the cornea, passes through the iris and is converged by the intraocular lens 20 on the macula. The optical zone portion 32 in this embodiment defines an area through which the light path intersects and passes through the anterior wall member 26 and the posterior wall member 28. The optical zone portion 32 may be commensurate with or smaller in width (that is, diameter) than the central cavity 26c. Alternatively, the intraocular lens may be implanted in the anterior chamber of the eye.

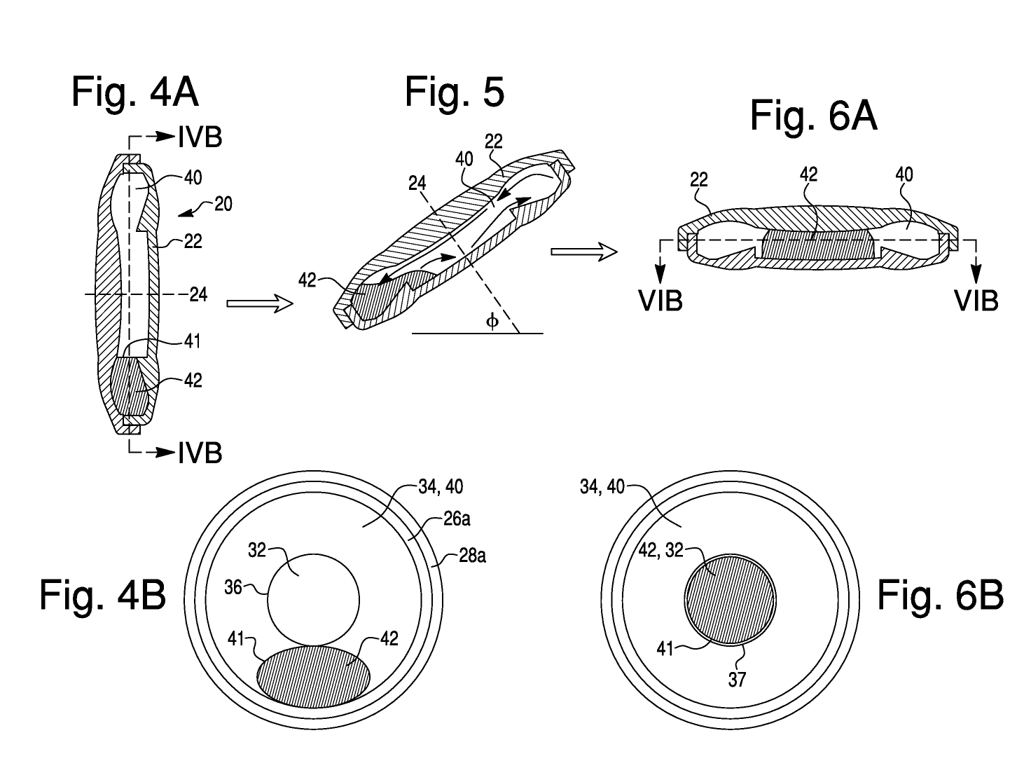

FIGS. 4A, 4B, 5, 6A, 6B, and 7-10 are schematics showing the chamber 30 of the lens body 22 of FIGS. 1-3 filled with an optically transmissive first fluid 40 and an optically transmissive second fluid 42. As shown, the fluids 40 and 42 are both depicted as liquids, and substantially no gas is contained in the chamber 30. In alternative embodiments, one of the fluids may constitute a gas or mixture of gases, or a vacuum. In the illustrated embodiment, the second fluid 42 has a higher density and a different refractive index than the first fluid 40. The first fluid 40 and the second fluid 42 are substantially immiscible with one another. The first and second fluids 40 and 42 contact one another at a contact interface 41.

FIGS. 4A and 4B show the intraocular lens 20 of the first embodiment positioned in a straight-ahead gaze position with the optical axis 24 horizontally oriented. FIG. 4B is a vertical cross-sectional view taken along sectional line IVB-IVB of FIG. 4A. As is understood in the art, the eye is not rotationally symmetric, so that the optical axis 24 and the visual axis are substantially but not perfectly co-linear. In the straight ahead gaze position of FIG. 4A, the second fluid 42 of higher density rests at the bottom of the chamber 30 in the non-optical zone 34. As best shown in FIG. 4B, in the straight-ahead gaze the second fluid 42 is positioned outside the cavity 26c and forms a “bubble” at the bottom of the chamber 30, below the annular shoulder 37. As shown in FIGS. 4A and 4B, in straight-ahead gaze the first fluid 40 is present in a sufficient amount to substantially fill the pocket 26c and the remainder of the non-optical zone portion 34 not filled by the second fluid 42. The first fluid in the optical portion 32 extends across the thickness of the chamber 30 so that the interior surfaces 26b and 28b of the optic elements of the anterior wall member 26 and the posterior wall member 28 contact the first fluid 40. The contact interface 41 is in the non-optical zone portion 34. Hence, in the straight-ahead gaze the second fluid 42 is not intersected by the optical axis 24, and vision is not affected by the refractive index of the second fluid 42 in the straight-ahead gaze.

As shown in FIGS. 5, 6A, and 6B, when the intraocular lens 20 is tilted forward, such as in the case of a patient or user having an implanted intraocular lens 20 tilting his or her head forward into a reading position, the optical axis 24 of the intraocular lens 20 eventually reaches an effective angle φ at which the second fluid 42 moves through the constricted passage, that is, over the ridge 36, into the central cavity 26c, where the second fluid 42 is substituted for the first fluid 40 in the optical zone portion 32. The second fluid 42 moves as a unitary mass or “bubble” from the non-optical zone portion 34 to the optical zone portion 32, similar to the principles by which a carpenter's or spirit level operates. The “bubble” of second fluid 42 desirably moves quickly, almost instantaneously from the non-optical zone portion 34 to the optical zone portion 32 when an effective tilt angle φ is reached. As best shown in FIG. 6A, the bubble of second fluid 42 bridges the gap between regions of the interior surfaces 26b, 28b corresponding to the optical zone portion 32.

The detainment structure 36 (embodied as a ridge in the first embodiment) and the shoulder 37 delay the onset of the fluid substitution so that the flow of second fluid 42 into the optical zone portion 32 starts at a greater angle φ than had the ridge 36 not been present. As discussed further below, the detainment structure 36 and the height of the shoulder 37 may be configured so that this effective angle φ coincides with a desired “reading position” for focusing light from the punctum proximum or pp onto the retina.

Once the effective tilt angle φ is reached and the second fluid 42 is transferred into the optical zone portion 32, the annular shoulder 37 defining the periphery of the central cavity 26c retains the second fluid 42 in the optical zone portion 32 through “reading” positions to a tilt angle of at least 90 degrees, as shown in FIGS. 6A and 6B. At the same time, the first fluid 40 is outside of the optical zone portion 32, i.e., in the non-optical zone portion 34, so as not to be along the optical axis and so that the refractive index of the first fluid 40 does not affect vision in the downward-gaze position. As best shown in FIG. 6B, the first fluid 40 concentrically surrounds the second fluid 42 in the downward gaze, with a substantially circular interface 41.

As best shown in FIG. 6A, the second fluid 42 substituted for the first fluid 40 in the optical zone portion 32 extends (or “bridges”) the gap between the interior surfaces 26b and 28b in the optical zone portion 32, without stacking on or below the first liquid 40. Without wishing to be bound by any theory, it is believed that the downward gaze substitution of the second liquid 42 for the first liquid 40 without stacking is due to the close proximity of the interior surfaces 26b and 28b to one another. The clearance between the interior surfaces 26b and 28b is insufficient to receive the curved interface 41 between the first and second fluids 40 and 42. This is believed to be due at least in part to surface tension. Hence, for the most part only one fluid 40 or 42 is received in the optical zone portion 42 at a time. (In FIGS. 6A and 6B, the amount of second fluid 42 is slightly less than the amount needed to completely fill the cavity 26c, and hence the contact interface 37 is present inside the cavity 26c. It may be desirable to include slightly more second fluid 42 in the chamber 30, and consequently slight less primary fluid 34, so that the second fluid 42 fills the central cavity 26c and the optical zone 32. The amount of second fluid 42 may match the volume of the central cavity 26c.) The optical axis 24 thus extends through only one of the fluids 40 or 42, depending upon the tilt angle (except for the brief instant during which fluid substitution takes place).

The spacing between the interior surfaces 26b and 28b in the portion of the chamber 30 corresponding to the optical zone 32 may be, for example, about 0.5 mm to about 1.5 mm, or about 1.25 mm to about 1.5 mm, with the intraocular lens 20 having an overall thickness (between opposite exterior surfaces of the anterior wall member 26 and the posterior wall member 28) of, for example, about 1.5 mm to about 3.5 mm, or about 1.5 to 2.2 mm, or about 1.5 mm to about 2.1 mm, or about 2.0 mm to about 2.2 mm. The diameter of the optical zone 32 and the cavity 26c may be, for example, about 3 mm. The lens 20 can be further tailored for individual users as needed or desired. For example, for optical zones 32 greater than 3 or 4 mm, it may be desirable to apply an annular opaque mask to eliminate optical aberrations that might otherwise arise if the subject's pupils are larger than the diameter of the optical zone 32.

The fluid substitution by which the second fluid 42 replaces the first fluid 40 in the cavity 26c takes place during downward tilting, that is, as a subject's head with the implanted intraocular lens 20 tilts downward from a straight forward position (FIG. 4A) into a reading position. The second fluid 42 remains in the optical zone 32 from an effective angle φ at which the fluid substitution takes place to at least 90 degrees. The effective angle φ shown in FIG. 5 is a measurement of the angular displacement of the optical axis 24 relative to horizontal. The effective angle at which fluid substitution takes place is greater than zero degrees and less than 90 degrees. That is, while FIG. 6 shows the second fluid 42 fully substituted into the optical zone portion 32 at the effective angle φ of 90 degrees, it is desirable in practice for the fluid substitution to initially take place at a lesser angle so that a person implanted with the intraocular lens 20 does not need to stare straight downward at 90 degrees in order to realize the short-distance or “reading” benefit of the bi-focal prosthesis. For example, it may be desirable for the fluid substitution to take place at an effective angle φ starting in a range of 20 to 70 degrees, 30 to 70 degrees, 30 to 60 degrees, or 40 to 50 degrees to provide the user with more comfortable reading angles that are less stressful on the user's neck.

The detainment structure 36 allows the fluid substitution to be delayed until a suitable effective angle is reached. Generally, smaller constrictions and “taller” detainment structures 36 will cause the fluid substitution to take place at a greater effective angle, i.e., the head must be tilted by a greater downward angle to cause fluid substitution for near-sight accommodation. After the fluid substitution occurs, the annular shoulder 37 surrounding the central cavity 26c stabilizes the second liquid 42 in the optical zone portion 32 so that near-sight vision is stabilized. The second liquid 42 remains in the optical zone portion 32 at downward angles in a range of the effective angle φ to at least 90 degrees.

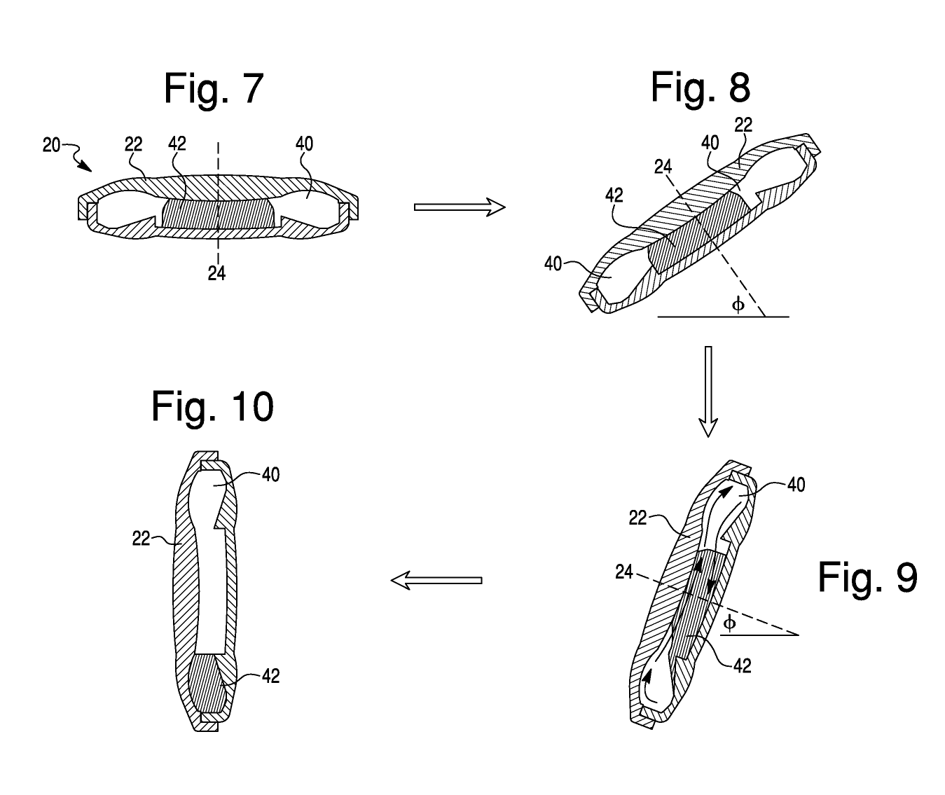

Fluid movement in the intraocular lens 20 during upward tilting movement, i.e., from a reading position to straight ahead gaze, will now be discussed in reference to FIGS. 7-10.

As shown in the downward gaze position of FIG. 7, the second fluid 42 is positioned in the optical zone portion 32, and the first fluid 40 is in the non-optical zone portion 34 annularly surrounding the second fluid 42. In the illustrated embodiment of FIG. 7, the user initially starts with his or her head arranged so that the optical axis 24 is φ=90 degrees. At this angle, the annular shoulder 37 surrounding the central cavity 26c retains the second fluid 42 in the optical zone portion 32, while the first fluid 40 is substantially outside of the optical zone portion 32, that is, in the non-optical zone portion 34 surrounding the second fluid 42 and the central cavity 26c. FIG. 8 shows the intraocular lens 20 with its optical axis at an angle φ of about 60 degrees, and the second fluid 42 retained in the optical zone portion 32. That is, the second fluid 42 remains captured in the optical zone portion 32 as the head lifts upward from FIG. 7 to FIG. 8 and the angle between the optical axis 24 and the horizontal decreases.

As shown in FIG. 9, gravity and the greater density of the second fluid 42 eventually overcome the capture-effect of the shoulder 37 and the delay effect of the detainment structure 36, and the fluid substitution reverses itself. That is, the first fluid 40 is returned to the central cavity 26c and the optical zone portion 32, and the second fluid 42 returns to the non-optical zone portion 34. As shown in FIG. 9, the onset of fluid substitution may start around an effective angle φ of about 30 degrees, for example. Preferably the fluid substitution occurs substantially instantaneously once the fluids 40 and 42 start to exchange places. After the reverse fluid substitution, the intraocular lens 20 focuses for distance vision through the first fluid 40 in the optical zone portion 32.

The particular effective angle φ at which fluid substitution begins may be controlled by manipulating the size of the height and shape of the detainment structure 36 and the volume and depth of the cavity 26c. Generally, the onset of the “reverse” fluid substitution shown in FIG. 9 during upward head movement may be delayed by providing the cavity 26c with a greater depth and/or by provision a narrower constricted passage established by the ridge 36. Conversely, the onset of the fluid substitution shown in FIG. 9 during upward head movement may be hastened (to occur at a greater effective angle φ) by providing the cavity 26c with a lesser depth and/or by constructing the detainment structure 36 to provide a greater (thicker) constriction between the optical zone portion 32 and the non-optical zone portion 34.

The curvatures of the optic elements of the anterior wall member 26 and the posterior wall member 28 and the refractive indices of the first and second fluids are selected to provide a desired overall power in straight ahead and down gaze. In one exemplary embodiment, the curvature of the optic elements of the anterior wall member 26 and the posterior wall member 28 (in the optical zone 32) and the selection of the first fluid 40 (with its refractive index) cause light rays traveling from the punctum remotum (pr) through the eye to focus on the macula. Similarly, the curvature of the optic elements of the anterior wall member 26 and the posterior wall member 28 (in the optical zone 32) and the selection of the second fluid 42 (with its refractive index) may be determined such that light traveling from the punctum proximum (pp) through the eye is focused on the macula for near vision. Adjustment of the lens power by modification of the optic body curvature is within the purview of those having ordinary skill in the art. Optical design tools such as Zemax® may be useful in optic design. In determining proper optics for focusing on the macula, consideration may be given to the initial refractive effect that the cornea has on incoming light rays.

By way of example, for refractive correction surgery, it is preferable to provide a power of about 12 and about 25 diopters in straight-ahead gaze (based on the number of diopters required to provide emmetropia), with the target typically being approximately 20 diopters. On down gaze, the prosthesis may be provided additional power, depending upon the intended application. For example, 1.0 to 4.0 diopter (e.g., 2.0 to 3.0 diopter) additional power may be suitable for treatment of presbyopia, while 4 to 12 diopter additional power may be useful for treating low vision patients. More or less additional power may be desirable, depending upon the patient. It is within the scope of the invention to form a lens which is capable of translating to additional desired power for accommodation of eyesight, whether more (+) power or more (−) power upon down gaze. Selection of appropriate fluids to obtain such power changes by fluid substitution can be determined with the assistance of Snell's Law and is based on the index of refraction (IR) of the fluid. “Near” vision may provide the desired amount of accommodation for focusing on an object at, for example, 3 to 9 inches from the eye.

The change in power of the intraocular lens 20 from “far” vision to “near” vision (and vice versa) is achieved by downward tilting movement without the need for convexity change (e.g., flexing) of the lens 20, and without moving the intraocular lens 20 relative to the eye structure, e.g., towards or away from the macula.

The first and second fluids are preferably optically transparent and substantially immiscible with one another. Although the term fluid as used herein may include a liquid or gas, the first and second fluids are preferably both liquids at ambient (room) temperature. Fluids that may be used in the chamber 30 of the lens body 22 include, but are not limited to, those common to ophthalmic surgery and that are non-hazardous. As noted above, in particularly exemplary embodiments the refractive indices of the first and second fluids differ from one another by an amount to produce an overall power increase of 1.0 to 4.0 diopter upon tilting downward.

The second fluid may have a combination of a low refractive index and high specific gravity compared to the first fluid. For example, the first fluid 40 may be silicone oil such as polydimethylsiloxane, polydimethyldiphenylsiloxane, etc., having a refractive index in the range of about 1.41 to about 1.48, and the second fluid 42 may be a perfluorocarbon having a refractive index in the range of about 1.33 to about 1.36. Generally speaking, a greater difference between the refractive indices of the first and second fluids allows the lens body 22 to be made thinner since less optic curvature is required.

The lens body 22 is preferably made of one or more materials biologically compatible with the human eye. In particular, the materials are preferably non-toxic, non-hemolytic, and non-irritant. The lens body 22 and haptics 38 are preferably made of a material that will undergo little or no degradation in optical performance over their intended period of use. For example, the lens body 22 may be constructed of rigid biocompatible materials, such as, for example, polymethylmethacrylate (PMMA), or flexible, deformable materials, such as silicones, hydrophobic acrylic polymers (e.g., copolymers/terpolymers: butylacrylate, ethylmethacrylate, fluorinated, aromatic monomers such as phenylethylmethacrylate), and the like which enable the lens body 22 to be rolled, deformed, or folded for insertion through a small incision into the eye. The above list is merely representative, not exhaustive, of the possible materials that may be used in this invention. The interior surfaces 26b and 28b of the lens body 22 may be coated with a low-friction material such as perfluorocarbon. Beneficially, it has been found that PMMA does not require the use of such coatings.

Methods of making lens bodies are well known in the art and are described throughout the literature. These methods, which are suitable for use with the various aspects of the present invention, include, not necessarily by limitation, molding (e.g., injection molding) and lathing. The formation of a molded body 22 with an internal chamber 30 is well known in the injection molding and lathing arts. Methods of gel-capsule manufacture as applied in the pharmaceutical industry may also be applied, as these methods describe introduction of fluids into capsules without leaving vacuum or air space within the capsule. As mentioned above, the anterior and posterior lens may be made as a unitary piece, or separately then joined together, such as by adhesive (UV cure epoxy adhesive), sealant, fusion, or the like.

The first and second fluids 40 and 42 may be introduced and retained in the chamber 30 prior to implanting or otherwise applying the prosthesis to an eye. The first and second fluids 40 and 42 may be introduced into the chamber by any technique consistent with the objects of this invention. For example, a syringe or the like may be used for injecting the fluids into the chamber. Optionally, an entry port may be provided in the optic body for introducing the fluids into the chamber 30 of the lens body 22. The entry port may be formed, for example, by injection molding, by penetrating the lens body 22 with a suitable hole-making instrument, such as a drill or needle, or by an injecting instrument, e.g., syringe, during introduction of the fluids. Other techniques may also be used to form the lens body 22.

The lens body 22 may include a vent port for expelling gas (usually air) from inside the chamber 30 as the fluids are introduced through the entry port. The vent may be separate from the entry port, or may be the same as the entry port such that gas entrapped in the chamber is expelled through the same port that the fluids are introduced into the chamber. Alternatively, the chamber may be evacuated prior to the introduction of the fluids. Subsequent to introducing the fluid into the chamber, the entry port and optional vent may be sealed to enclose the chamber in a known manner, such as by fusion or plugging with a compatible material, which may be the same or different than the material of which the lens body 22 is made.

In an exemplary embodiment, the prosthesis can be inserted into the posterior chamber of the human eye, such as into the capsular bag posterior to the iris to replace the physiological (natural) lens in the capsular bag positioned using known equipment and techniques. By way of example, intra-capsular cataract extraction and IOL implantation utilizing clear corneal incision (CCI), phacoemulsification or similar technique may be used to insert the intraocular lens after the physiological crystalline lens has been removed from the capsular bag. The incision into the eye may be made by diamond blade, a metal blade, a light source, such as a laser, or other suitable instrument. The incision may be made at any appropriate position, including along the cornea or sclera. It is possible to make the incision “on axis”, as may be desired in the case of astigmatism. Benefits to making the incision under the upper lid include reduction in stitching, greater cosmetic appeal, and reduced recovery time for wound healing. The prosthesis is optionally rolled or folded prior to insertion into the eye, and may be inserted through a small incision, such as on the order of about 3 mm. It is to be understood that as referred to herein, the term “capsular bag” includes a capsular bag having its front surface open, torn, partially removed, or completely removed due to surgical procedure, e.g., for removing the physiological lens, or other reasons.

Although the prosthesis has been described above as an intraocular lens for implantation, it should further be understood that prosthesis may be an exterior device applied outside of the eye, for example, mounted on frames or eyeglasses in front of eye or in a contact lens. The prosthesis may be used in combination with a physiological or synthetic lens placed in the anterior and/or posterior chamber(s). An external prosthesis may have greater dimensions than described above, because an external prosthesis need not implantable into eye.

The prosthesis can be used for various eye conditions and diseases, including, for example, presbyopia, aphakia, pseudophakia, anterior cortical cataract extraction (acce), posterior cortical cataract extraction (pcce), and the like. Of particular interest yet not necessarily by limitation, the intraocular lens of embodiments described herein is useful for treating retinal degenerative conditions (or “low vision”), and more particularly for reducing the effects of a scotomatous area on a visual field of a person having a retinal degenerative condition.

Treatment of RDCs may be accomplished by designing the prosthesis of the present invention as a Galilean-type device, wherein an objective lens is positioned in front of the intraocular lens to establish a telescopic benefit and a near-magnifying benefit. The telescopic benefit is derived from the effective power of the intraocular lens being calculated to be negative in power, and the objective lens in front of the intraocular lens being calculated to be positive in power. The focal points and/or focal planes of the objective and intraocular lenses may be coincident with one another, as is the case in a Galilean telescopic system. The combination of the negative intraocular lens and positive objective lens of prosthesis creates a telescopic power of a Galilean type, provided the focal planes of intraocular and objective are coincident. As referred to herein and generally understood in the art, a “negative power” lens is a “diverging lens”, i.e., a lens having a cumulative effect of diverging light passing through the lens. On the other hand, a “positive power” lens is a “converging lens”, i.e., a lens having a cumulative effect of converging light rays passing through the lens. The power of the prosthesis is controlled through selection of the fluids and lens curvatures. By controlling the negative power of the ocular lens and the positive power of the objective lens, a desired magnification can be obtained. In the straight-ahead gaze, the overall telescopic effect of the ocular and objective lens preferably is negative. In the downward gaze, the prosthesis provides a near point Galilean low vision magnifier.

The telescopic effect of this embodiment can reduce the effects of a scotomatous area of an individual afflicted with a RDC in straight ahead and down gazes. Without wishing to necessarily be bound by any theory, it is believed that the telescopic optics established by embodiments particularly useful in the treatment of RDCs enlarge the image desired to be viewed beyond the borders of the damaged region of the retina (and more particularly the macula) which is responsible for the scotoma, into healthy areas of the retina. As a consequence, although the scotomatous area is not removed from the field of vision, the viewed object is shifted, magnified, or otherwise moved so that a greater percentage of the object is viewed outside of the scotoma. Reversing the optics of a Galilean magnifier expands a user's field of view, which is particularly useful for treatment of conditions that restrict the user's field of view, such as glaucoma and retinitis pigmentosa (RP).

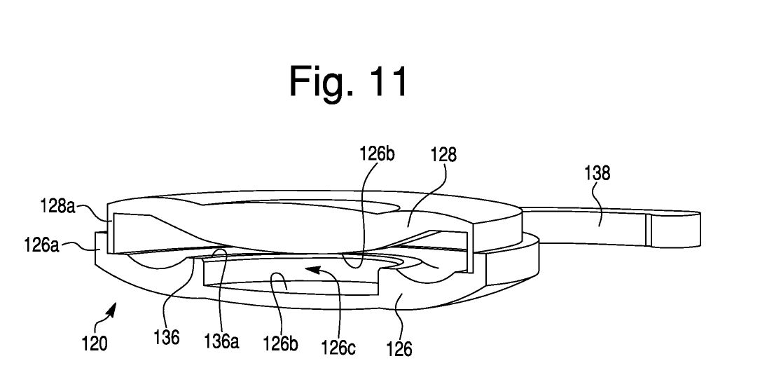

It should be understood that modifications and variations are possible to the embodiments described above. For example, FIG. 11 is a cut-away isometric view of a multi-focus intraocular prosthesis according to another embodiment of the invention in which like parts to the above embodiment of FIGS. 1-10 are designated with like reference numerals, except for the addition of the prefix “1” so that the reference numerals of FIG. 11 are in the one hundreds. The detainment structure 136 includes a beveled edge 136 at the outer periphery of the pocket 126c.

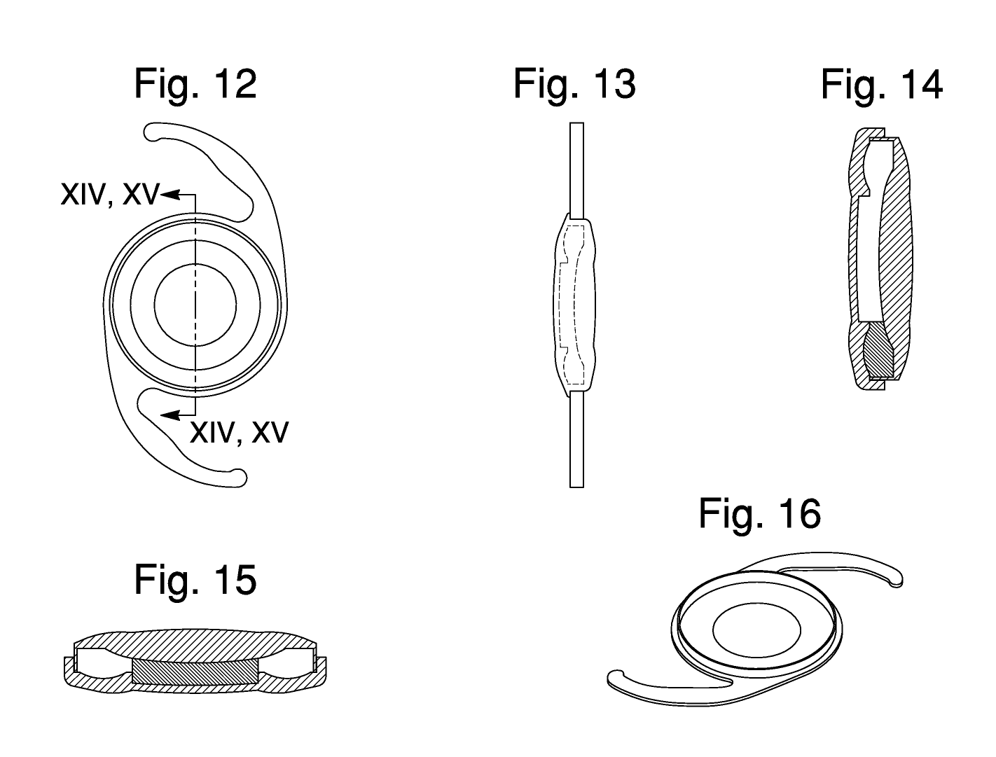

Another embodiment is shown in FIGS. 12-16 in which the interior surfaces have different curvatures than the interior surfaces 26b, 28b of the above embodiment of FIGS. 1-10.

FIGS. 17A, 17B, 18, 19A, and 19B show a multi-focus intraocular prosthesis according to another embodiment of the invention in which like parts to the above embodiment of FIGS. 1-10 are designated with like reference numerals, except for the addition of the prefix “2” so that the reference numerals of FIGS. 16-19 are in the two hundreds. In this embodiment, the second fluid 242 has a lower density and a different refractive index than the first fluid 240. The central cavity or pocket is shown formed in the inner surface of the posterior wall member.

FIGS. 17A and 17B show the intraocular lens 220 of the embodiment positioned in a straight-ahead gaze position with the optical axis 224 horizontally oriented. FIG. 17B is a vertical cross-sectional view taken along sectional line IVB-IVB of FIG. 17A. As is understood in the art, the eye is not rotationally symmetric, so that the optical axis 224 and the visual axis are substantially but not perfectly co-linear. In the straight ahead gaze position of FIG. 17A, the second fluid 242 of lower density rests at the top of the chamber in the non-optical zone 234. As best shown in FIG. 17B, in the straight-ahead gaze the second fluid 242 is positioned outside the cavity and forms a “bubble” at the top of the chamber, below the annular shoulder 237. In straight-ahead gaze the first fluid 240 is present in a sufficient amount to substantially fill the pocket and the remainder of the non-optical zone portion 234 not filled by the second fluid 242. The first fluid in the optical portion 232 extends across the thickness of the chamber so that the interior surfaces of the optic elements of the anterior wall member 226 and the posterior wall member 228 contact the first fluid 240. The contact interface 241 is in the non-optical zone portion 234. Hence, in the straight-ahead gaze the second fluid 242 is not intersected by the optical axis 224, and vision is not affected by the refractive index of the second fluid 242 in the straight-ahead gaze.

As shown in FIGS. 18, 19A, and 19B, when the intraocular lens 220 is tilted forward, such as in the case of a patient or user having an implanted intraocular lens 220 tilting his or her head forward into a reading position, the optical axis 224 of the intraocular lens 20 eventually reaches an effective angle φ at which the second fluid 242 moves through the constricted passage, that is, over the ridge and into the central cavity, where the second fluid 242 is substituted for the first fluid 240 in the optical zone portion 232. The second fluid 242 moves as a unitary mass or “bubble” from the non-optical zone portion 234 to the optical zone portion 232, similar to the principles by which a carpenter's or spirit level operates. The “bubble” of second fluid 242 desirably moves quickly, almost instantaneously from the non-optical zone portion 234 to the optical zone portion 232 when an effective tilt angle φ is reached. As best shown in FIG. 19A, the bubble of second fluid 242 bridges the gap between regions of the interior surfaces corresponding to the optical zone portion 232.

The detainment structure 236 (embodied as a ridge) and the shoulder 237 delay the onset of the fluid substitution so that the flow of second fluid 242 into the optical zone portion 232 starts at a greater angle φ than had the ridge 236 not been present. As discussed further below, the detainment structure 236 and the height of the shoulder 237 may be configured so that this effective angle φ coincides with a desired “reading position” for focusing light from the punctum proximum or pp onto the retina.

Once the effective tilt angle φ is reached and the second fluid 242 is transferred into the optical zone portion 232, the annular shoulder 237 defining the periphery of the central cavity retains the second fluid 242 in the optical zone portion 232 through “reading” positions to a tilt angle of at least 90 degrees, as shown in FIGS. 19A and 19B. At the same time, the first fluid 240 is outside of the optical zone portion 232, i.e., in the non-optical zone portion 234, so as not to be along the optical axis 224 and so that the refractive index of the first fluid 240 does not affect vision in the downward-gaze position. As best shown in FIG. 19B, the first fluid 240 concentrically surrounds the second fluid 242 in the downward gaze, with a substantially circular interface 241.

As best shown in FIG. 19A, the second fluid 242 substituted for the first fluid 240 in the optical zone portion extends (or “bridges”) the gap between the interior surfaces in the optical zone portion, without stacking on or below the first liquid 240. Without wishing to be bound by any theory, it is believed that the downward gaze substitution of the second liquid 242 for the first liquid 240 without stacking is due to the close proximity of the interior surfaces to one another. The clearance between the interior surfaces is insufficient to receive the curved interface 241 between the first and second fluids 240 and 242. This is believed to be due at least in part to surface tension. Hence, for the most part only one fluid 240 or 242 is received in the optical zone portion 242 at a time. (In FIGS. 19A and 19B, the amount of second fluid 242 is slightly less than the amount needed to completely fill the cavity, and hence the contact interface 237 is present inside the cavity. It may be desirable to include slightly more second fluid 242 in the chamber, and consequently slight less primary fluid 234, so that the second fluid 242 fills the central cavity and the optical zone 232. The amount of second fluid 242 may match the volume of the central cavity.) The optical axis 224 thus extends through only one of the fluids 240 or 242, depending upon the tilt angle (except for the brief instant during which fluid substitution takes place).

The fluid substitution by which the second fluid 242 replaces the first fluid 240 in the cavity takes place during downward tilting, that is, as a subject's head with the implanted intraocular lens 220 tilts downward from a straight forward position (FIG. 19A) into a reading position. The second fluid 242 remains in the optical zone 232 from an effective angle φ at which the fluid substitution takes place to at least 90 degrees. The effective angle φ shown in FIG. 18 is a measurement of the angular displacement of the optical axis 224 relative to horizontal. The effective angle at which fluid substitution takes place is greater than zero degrees and less than 90 degrees. That is, while FIG. 18 shows the second fluid 242 fully substituted into the optical zone portion 232 at the effective angle φ of 90 degrees, it is desirable in practice for the fluid substitution to initially take place at a lesser angle so that a person implanted with the intraocular lens 220 does not need to stare straight downward at 90 degrees in order to realize the short-distance or “reading” benefit of the bi-focal prosthesis. For example, it may be desirable for the fluid substitution to take place at an effective angle φ starting in a range of 20 to 70 degrees, 30 to 70 degrees, 30 to 60 degrees, or 40 to 50 degrees to provide the user with more comfortable reading angles that are less stressful on the user's neck.

The detainment structure 236 allows the fluid substitution to be delayed until a suitable effective angle is reached. Generally, smaller constrictions and “taller” detainment structures 236 will cause the fluid substitution to take place at a greater effective angle, i.e., the head must be tilted by a greater downward angle to cause fluid substitution for near-sight accommodation. After the fluid substitution occurs, the annular shoulder 237 surrounding the central cavity stabilizes the second liquid 242 in the optical zone portion 32 so that near-sight vision is stabilized. The second liquid 242 remains in the optical zone portion 232 at downward angles in a range of the effective angle φ to at least 90 degrees.

The foregoing detailed description of the exemplary embodiments of the invention has been provided for the purposes of illustration and description, and is not intended to be exhaustive or to limit the invention to the precise embodiments disclosed. The embodiments were chosen and described in order to best explain the principles of the invention and its practical application, thereby enabling others skilled in the art to understand the invention for various embodiments and with various modifications as are suited to the particular use contemplated. It is intended that the scope of the invention cover various modifications and equivalents included within the spirit and scope of the appended claims.

What is claimed is:

1. A multi-focus intraocular lens comprising:
a lens body having a chamber;
first and second fluids in the chamber, wherein tilting movement of the lens body induces fluid substitution between the first and second fluids in an optical zone portion of the lens body.

2. The multi-focus intraocular lens of claim 1, wherein the first fluid is silicone oil and the second fluid is perfluorocarbon.

3. A multi-focus intraocular prosthesis, comprising:
a lens body configured for placement in an eye to replace or supplement a physiological or artificial lens, the lens body having an optical axis and comprising a transparent anterior wall member and a transparent posterior wall member, the anterior wall member and the posterior wall member having respective inner surfaces that collectively establish a chamber within the lens body, the chamber comprising an optical zone portion intersected by the optical axis, a substantially annular non-optical zone portion peripherally arranged relative to the optical zone portion and in fluid communication with the optical zone portion, and a detainment structure; and
a plurality of fluids contained in the chamber, the plurality of fluids comprising a first fluid having a first refractive index and a first specific density and a second fluid having a second refractive index and a second specific density that differ from the first refractive index and the first specific density, respectively, the first and second fluids being immiscible with one another,
wherein the first fluid substantially fills the optical zone portion and the second fluid is situated substantially outside of the optical zone portion in the non-optical zone portion at a straight ahead gaze position in which the optical axis is horizontal, and wherein the second fluid substantially fills the optical zone portion and the first fluid is situated substantially outside of the optical zone portion in the non-optical zone portion at a downward gaze position in which the optical axis is at a tilt angle relative to horizontal of greater than zero but less than 90 degrees.

4. The multi-focus intraocular prosthesis of claim 3, wherein the detainment structure delays an onset of fluid substitution of the second fluid into the optical zone portion in exchange for the first fluid during downward tilting movement from the straight ahead gaze position to the downward gaze position.

5. The multi-focus intraocular prosthesis of claim 3, wherein the second fluid in the chamber has a volume substantially equal to the volume of the optical zone portion.

6. The multi-focus intraocular prosthesis of claim 5, wherein in the downward gaze position the first fluid substantially fills the non-optical zone portion to surround the second fluid substantially filling the optical zone portion.

7. The multi-focus intraocular prosthesis of claim 3, wherein the tilt angle at which the second fluid substantially fills the optical zone portion is greater than 20 degrees and less than 70 degrees.

8. The multi-focus intraocular prosthesis of claim 3, wherein the tilt angle at which the second fluid substantially fills the optical zone portion is greater than 30 degrees and less than 70 degrees.

9. The multi-focus intraocular prosthesis of claim 3, wherein the detainment structure is substantially annular and surrounds the optical zone portion.

10. The multi-focus intraocular prosthesis of claim 3, wherein the detainment structure is integral with and constitutes part of the inner surface of the anterior wall member.

11. The multi-focus intraocular prosthesis of claim 10, wherein the inner surface of the anterior wall member in a region corresponding to the optical zone portion is recessed into the anterior wall member relative to the detainment structure to form a central cavity.

12. The multi-focus intraocular prosthesis of claim 3, wherein the detainment structure is integral with and constitutes part of the inner surface of the posterior wall member.

13. The multi-focus intraocular prosthesis of claim 12, wherein the inner surface of the posterior wall member in a region corresponding to the optical zone portion is recessed into the posterior wall member relative to the detainment structure to form a central cavity.

14. The multi-focus intraocular prosthesis of claim 3, wherein the first refractive index and the second refractive index differ from one another by an amount to produce an overall power increase upon tilting downward.

15. The multi-focus intraocular prosthesis of claim 3, wherein the detainment structure comprises an annular ridge surrounding the optical zone portion.

16. The multi-focus intraocular prosthesis of claim 3, wherein the first fluid is silicone oil and the second fluid is perfluorocarbon.

17. The multi-focus intraocular prosthesis of claim 3, wherein fluid substitution of the second fluid for the first fluid in the optical zone portion is substantially instantaneous at the tilt angle.

18. The multi-focus intraocular prosthesis of claim 3, wherein in the straight ahead gaze position the first fluid bridges the gap between the inner surfaces of the anterior and posterior wall members in the optical zone portion, and wherein in the downward gaze position the second fluid bridges the gap between the inner surface of the anterior and posterior wall members in the optical zone portion.

19. A multi-focus intraocular prosthesis, comprising:
a lens body configured for placement in an eye to replace or supplement a physiological or artificial lens, the lens body having an optical axis and comprising a transparent anterior wall member and a transparent posterior wall member, the anterior wall member and the posterior wall member having respective inner surfaces that collectively establish a chamber within the lens body, the chamber comprising an optical zone portion intersected by the optical axis, and a substantially annular non-optical zone portion peripherally arranged relative to the optical zone portion and in fluid communication with the optical zone portion; and
a plurality of fluids contained in the chamber, the plurality of fluids comprising a first fluid having a first refractive index and a first specific density and a second fluid having a second refractive index and a second specific density that differ from the first refractive index and the first specific density, respectively, the first and second fluids being immiscible with one another,
wherein the first fluid substantially fills the optical zone portion and the second fluid is situated substantially outside of the optical zone portion in the non-optical zone portion at a straight ahead gaze position in which the optical axis is horizontal, wherein the second fluid substantially fills the optical zone portion and the first fluid is situated substantially outside of the optical zone portion in the non-optical zone portion at a downward gaze position in which the optical axis is at a tilt angle relative to horizontal of greater than zero but less than 90 degrees, and wherein fluid substitution of the second fluid for the first fluid in the optical zone portion occurs at the tilt angle.

20. The multi-focus intraocular prosthesis of claim 19, wherein fluid substitution is substantially instantaneous.

21. The multi-focus intraocular prosthesis of claim 19, wherein in the straight ahead gaze position the first fluid bridges the gap between the inner surfaces of the anterior and posterior wall members in the optical zone portion, and wherein in the downward gaze position the second fluid bridges the gap between the inner surface of the anterior and posterior wall members in the optical zone portion.
